(19) United States
(12) Patent Application Publication
(10) Pub. No.: US 2002/0132013 A1
(43) Pub. Date: Sep. 19, 2002

(54) Liquid cautery catheter

(76) Inventor: Harry Moulis, Ormond Beach, FL (US)

Correspondence Address: VAN DYKE & ASSOCIATES, P.A., 1630 HILLCREST STREET, ORLANDO, FL, 32803, US

(21) Appl. No.: 09/899,556

(22) Filed: Jul. 5, 2001

Related U.S. Application Data
(63) Continuation-in-part of application No. 09/882,811, filed on Jun. 18, 2001.
Application No. 09/882,811 is a continuation-in-part of application No. 09/808,368, filed on Mar. 14, 2001.

(51) Int. Cl. A61M 5/32; A61K 33/38; A61K 33/34; A61K 31/115; A61K 33/32; A61K 31/045

(52) U.S. Cl. 424/619; 424/637; 424/640; 514/694; 514/724; 514/731; 604/272

(57) ABSTRACT

Disclosed herein are novel cautery devices, and methods and kits implementing the same. The disclosed devices are especially useful for localized delivery of a liquid caustic agent to treat various defects associated with malformations and injuries resulting in chronic or acute bleeding, or to ablate tumors, occlude fistulae or other luminal structures. The disclosed devices have uses in a number of medical disciplines, and specific examples are provided pertaining to treatment of defects, malformations, and injuries, or bleeding due to medical procedures, in and along the gastrointestinal tract.

### BACKGROUND OF THE INVENTION

There are a number of vascular malformations, defects, or injuries that commonly occur along the lining of the intestine, parts of the gastrointestinal tract or urogenital pathways. Some of the more common types include angiodysplasias or telangiectasias (esophageal, gastric, duodenal, jejunal, ileal, colonic, rectal; Helmrich et al., Southern Medical Journal 83:1450-1453 (1990)), watermelon stomach (Gretz and Achem, Am. J Gastroentero. 93:890-895 (1998); Binmoeller and Lieberman, Gastrointest Endosc 37:192-193 1991);, gastric antral vascular ectasias, and radiation injury (radiation proctitis, esophagitis, gastritis, enteritis). A typical characteristic of these types of disorders is undesired bleeding (Lewis, Gastroenterology Clinics of North America 23:67-91; and Jaspersen et al., Gastrointest Endosc 40:40-44 (1994)). Indeed, gastrointestinal bleeding accounts for at least 2% of all hospital admissions each year (Levy, N. Engl. J. Med 290:1158 (1974)).

Conventional treatment of the foregoing disorders includes thermal treatment (Jensen et al. Gastointest Endosc 45:20-25 (1997); Askin and Lewis, Gastrointest Endosc 43:580583 (1996), Argon Plasma coagulation (Wahab et al., Endoscopy 29:176-181 (1997), direct pressure (Kirollos, M. J. Urology, 1998 August 160:477-478; Hirokazu, T. J. Urology, 1998 November 160,:1803); and/or laser treatment (Taylor et al, Gastrointest Endosc 52:353:357 (2000)). However, these conventional methods are not without their drawbacks. The medical equipment is relatively costly and can be cumbersome to use. Furthermore, they present the potential risks of perforation (Pierzchajilo, Colonoscopy, 22:451-470 (1995); Bedford et al., Am J Gastroenterol., 87:244-247 (1987)), or in the case of thermal treatment, heart disrythmias or even colonic explosions (Monahan et al, Gastrointest Endosc 38:40-43 (1992); Vellar et al., Br. J. Surg. 73:157-158 (1986); Donato and Memeo, Dis Colon Rectum 36:291-292 (1993); Shinagawa et al., Br. J. Surg. 72:306 (1985)). Argon plasma coagulation has been shown to cause inflammatory polyps (Schmeck-Lindenau and Heine, Endoscopy 30:93-94 (1998). Further, direct pressure may be insufficient to achieve lasting hemostasis (Hirokazu, T. .J Urology, 1998 Nov. 160:1803).

U.S. Pat. Nos. 6,187,346 and 6,165,492 to Neuwirth et al. disclose chemical cauterization devices and methods used for treatment of lesions occurring in the uterus. The system taught in these patents involves filling the uterus with a caustic agent, such as silver nitrate, and then neutralizing the cauterizing agent with a sodium chloride solution. However, the methods taught in U.S. Pat. Nos. 6,187,346 and 6,165,492 are not applicable to situations where filling a cavity, such as a uterine cavity, is not possible. Furthermore, these patents do not teach devices that control delivery of a caustic agent as to allow for focal treatment of a limited area of tissue.

In view of the problems associated with traditional treatments, there is a need in the art for a cautery method that overcomes these problems, and provides an easy to use, inexpensive system for cauterization. While gastroenterologists encounter a number of chronic bleeding disorders, other medical disciplines, such as otorhinolaryngology, pulmonology, gynecology, urology, general surgery, thoracic surgery, and orthopedic surgery, may encounter deformations, defects, and/or injuries that result in undesired bleeding as well. Ideally, the new cautery method would be readily adaptable for use in medical procedures in the GI tract but also other organ systems. For example, transurethral resection of the prostate, or retropubic protatectomy may lead to massive bleeding and an inability to achieve hemostasis in some patients (Touyama H. J. Urology, 1998 November 160:1803; Kirollos. M. J. Urology 1998 August 160:477-478). Studies related to these types of surgeries have discussed the problems of severe intra-operative bleeding. In most instances arterial bleeding can be controlled through electrocoagulation, whereas venous bleeding can be controlled by placing the catheter on traction and over-inflating the catheter balloon to create pressure sufficient to stop bleeding and promote coagulation. However, not all bleeding is of arterial origin and catheter traction to reduce post-operative venous bleeding only works when applied, having no effect after removal. (Walker E M. et al Br. J Urol 1995 May: 75(5):614-7) In those patients with severe arteriovenous malformation, these procedures are insufficient to achieve complete hemostasis, leading to continued blood loss which may become life threatening. (Touyama, H. J. Urology 1998 November 160:1803). The double lumen catheter of the present invention solves this problem.

### SUMMARY OF THE INVENTION

The subject invention is directed to a novel cautery system which provides localized cauterization and is easily adaptable for implementation in a number of surgical and non-surgical procedures. Specifically exemplified is a cautery system that delivers a liquid caustic agent to a site of need, wherein the liquid caustic agent is administered through the use of a catheter or other similar device. According to one aspect, the subject invention pertains to a catheter that has a regulating tip at one end, wherein the regulating tip has an impeder, such as, e.g., a sponge, fritted glass or other porous material disposed therein. As the tip contacts, or is placed proximate to, a site of need, a controlled amount of the caustic agent is released. Alternatively, the regulating tip has other configurations to allow for the controlled delivery of the caustic agent, such as the provision of a barrier having one or more small holes. The regulating tip enables controlled, focal delivery of the caustic agent whereby contact with non-target areas is avoided or limited.

In a further embodiment, the subject catheter comprises at least one first conduit for transporting a cauterizing agent and at least one second conduit attached to said first conduit for transporting air. The catheter is designed to have an external diameter appropriate for use. In one specific embodiment, a single lumen catheter is presented wherein cauterizing agent is transported to a contact site as described above. In another embodiment, a multiple lumen catheter is presented wherein a first lumen defines a conduit for transporting a cauterizing agent to a contact site as described above, and a second lumen which defines an air conduit attached to a inflatable balloon for use as a positioning and anchoring device.

At the other end of the catheter, opposite to the regulating tip, the single or multiple lumen catheter is connected to a container for storing and supplying the caustic agent. A preferred container is a syringe comprising a plunger, barrel and a connecting end. More preferably, the catheter is equipped with an attachment means such as a female or male luer-lok end, which readily attaches to a syringe comprising the caustic agent. In a double lumen design, the second lumen is connected to an air pressure device. A preferred device is a syringe comprising a plunger, barrel filled with air and a connecting end. Those skilled in the art will appreciate that more than one lumen for each purpose, i.e., transporting a cauterizing agent and transporting air for filling an inflatable chamber, may be employed.

According to a further aspect, the subject invention pertains to a method of delivering a caustic agent utilizing the cautery device of the subject invention. The subject method can be used to treat various malformations, defects, and injuries, while preventing chronic blood loss associated with surgery

In another aspect, the subject invention pertains to a non-surgical, non-steroidal method of contraception.

In yet another aspect, the subject invention pertains to a kit comprising one or more syringes, a single lumen catheter, multiple lumen catheters, or both and a volume of a caustic agent, preferably provided in a separate container.

These and other advantageous aspects of the subject invention will be described in further detail below.

### DESCRIPTION OF THE DRAWINGS

FIG. 1 shows a first embodiment of the subject invention that comprises a single lumen catheter connected to a syringe containing a caustic agent.

FIGS. 2a-d show magnified views of four alternate embodiments of the tip of the catheter shown in FIG. 1.

FIG. 3a shows a second embodiment of the subject invention that comprises a double lumen catheter connected to a first syringe containing caustic agent and a second syringe filled with air.

FIG. 3b shows a close-up cross-sectional view across the top of the double lumen catheter showing the lumen through which air or caustic agent is delivered, and showing pores through which a caustic agent may extrude radially.

FIG. 3c shows a close-up cross-sectional view of the double lumen catheter modified such that both lumens are contained within the outer circumference of the catheter, and showing pores through which a caustic agent may extrude radially.

### DESCRIPTION OF THE PREFERRED EMBODIMENTS

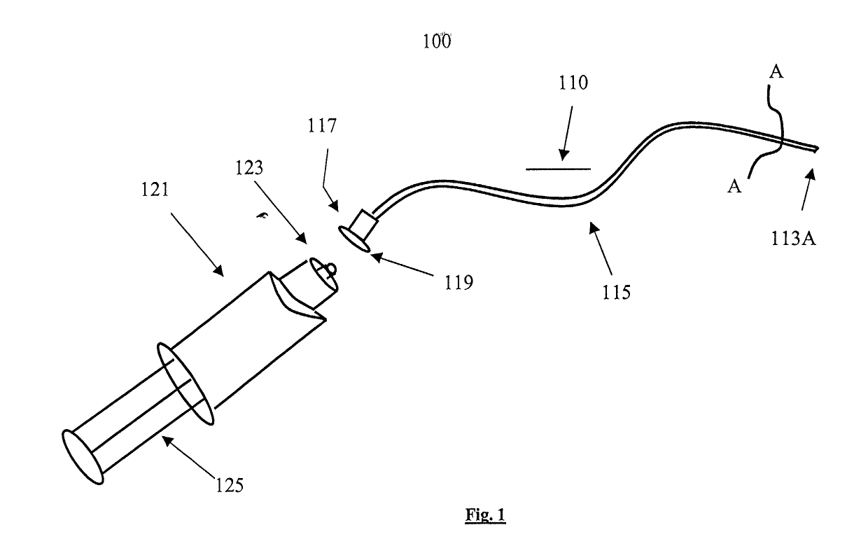

As discussed above, the subject invention is directed to medical devices useful as a cautery, and specifically for delivering a caustic agent to a site of need. Turning to the drawings, FIG. 1 shows an embodiment of a single lumen cautery device 100 that comprises a flexible catheter 110. The catheter 110 has a first end 113A (out of which a caustic agent is delivered), a flexible, elongated portion 115, and second end 117 that has a female luer-lock 119 connector disposed thereon for attaching to a male luer-lock end 123 of a syringe 121. During typical use of the cautery device 100, the syringe 121 is provided with an amount of a caustic agent and attached to the female luer-lock connector 119. The first end 113A and elongated portion 115 of the catheter 110 are guided through an endoscope and positioned proximate to site of need. By applying appropriate pressure to the plunger 125 of the syringe 121, the caustic agent travels through the catheter 110 and is ejected out at the first end 113A and onto the site of need in a controlled manner.

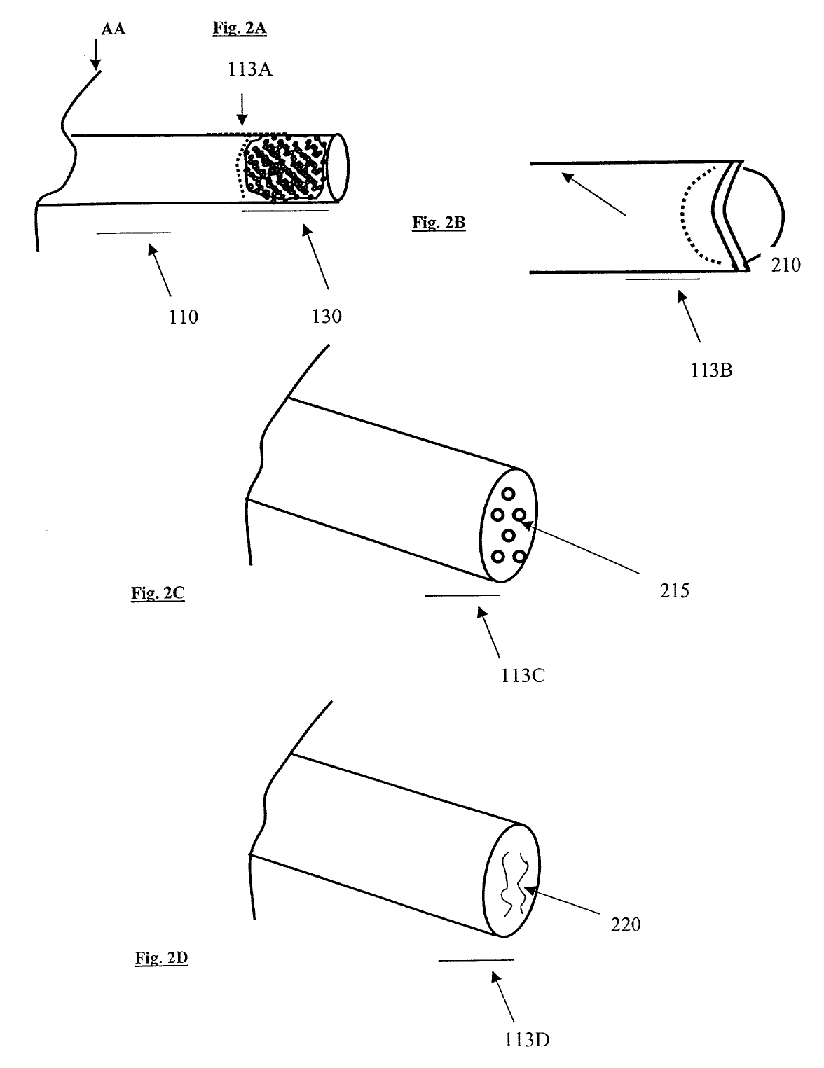

Shown in FIG. 2A is close-up depiction of the first end 113A of the catheter 110 up from the break line AA. FIG. 2A illustrates the placement of a permeable material 130 (fritted glass, sponge, etc.) in the first end 113A, which governs the delivery of the caustic agent out of the catheter 110. The provision of the liquid permeable material 130 prevents uncontrolled spilling and flow of the caustic agent out of the first end 113A, thereby limiting contact of the caustic agent with surrounding tissues. The permeable material 130 is preferably a sponge, fritted glass, or a semi-permeable membrane. Those skilled in the art, in view of the teachings herein, will readily appreciate that various materials can be used to make the permeable material. FIGS. 2B,C and D show alternative embodiments of the first end of the catheter, which allows for controlled delivery of the caustic agent. FIG. 2B shows an embodiment that comprises a roll-on ball 210 attached to a first end 113B for applying caustic agent to the site of need. FIG. 2C shows an embodiment which comprises a closed first end 113C with a plurality of perforations 215 out of which caustic agent is ejected. FIG. 2D shows an embodiment that has a semi-permeable membrane 220 rigidly attached to a first end 113D. Alternatively, the end is formed from rubber or other elastic material and is configured to apply a more viscous caustic agent, as well as an agent which immediately hardens upon contact with fluids. For example, the end can comprise a rubber tip that has a slit formed thereon, e.g., such as conventional rubber tips for applying glue. When pressure is applied to the syringe plunger, the caustic agent is ejected and applied to the site of need. This type of end is directed toward avoiding clogging of the end. Those skilled in the art will appreciate that other types of ends can be configured depending on the viscosity and drying time of the caustic agent. Examples of tips known in the art which could be adapted for use in accord with the subject invention include, but are not limited to, U.S. Pat. Nos. 5,199,808; 4,480,940; 4,050,826; 2,998,616; and 5,568,990.

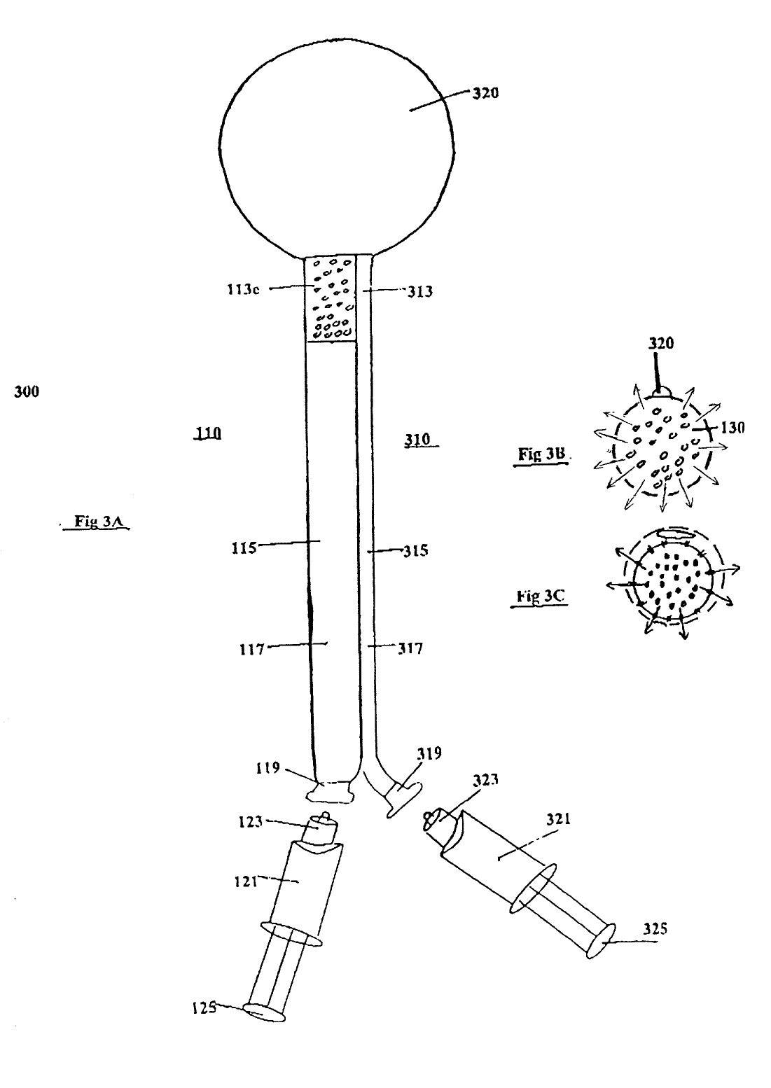

FIG. 3A shows a double lumen catheter of the present invention, generally indicated at 300, which is substantially similar in function and design to that described in FIG. 1, but which incorporates a single catheter having two lumens and associated components. The device comprises a first catheter 110 having a first end 113E out of which a caustic agent is delivered, a flexible, elongated portion 115, and second end 117 that has a female luer-lock 119 connector disposed thereon for attaching to a male luer-lock end 123 of a syringe 121 having first plunger 125. A second catheter 310 has a first end 313 terminating in a small balloon 320 which may be inflated or deflated, a flexible, elongated portion 315, and second end 317 that has a female luer-lock 319 connector disposed thereon for attaching to a male luer-lock end 323 of an air-filled syringe 321. The catheters are affixed together to allow simultaneous movement within a cavity. During typical use of the double lumen catheter device, both first and second catheters are guided through an endoscope such that the first end 113E of the first catheter 110 and the first end 313 of the second catheter 310 are positioned immediately above a site of need in a cavity adjacent to a tubular lumen. The syringe 121 of the first catheter 110 is provided with an amount of a caustic agent and attached to the female luer-lock connector 119. The syringe 321 attached to the second catheter 310 is filled with air and attached to the female luer-lock connector 319. A user compresses a second plunger 325 to force air from the syringe 321, through the catheter 310, and into the balloon 320 for inflation. When inflated the balloon 320 serves as an anchor by pressing against walls of a cavity, thereby holding the device in place for focal release of caustic agent. A user then compresses first plunger 125 to force caustic agent through the catheter 110 which is subsequently ejected out at the side at the first end 113E and onto the site of need in a controlled manner. Once the caustic agent has sufficiently reacted with tissue to alleviate bleeding, the balloon is deflated by releasing a second plunger 325, and withdrawing the catheter. FIG. 3B depicts a cross-sectional view from the first end of both portions of the double lumen catheter. The first end 113E of the first catheter 110 containing the permeable material 130 connected to the first end 313 of the second catheter 310 containing a balloon 320 (depicted in a deflated state). The first end 113E is slightly modified from 113A-D (see FIGS. 2 A-D) by allowing radiating extrusion, i.e. 0° to 360° of cauterizing agent out of the catheter. Those skilled in the art will appreciate that this end can also be used for the single lumen embodiment shown in FIG. 1. This end is especially adapted for the efficient, but focused treatment of an inner area of a small lumen or cavity. FIG. 3C shows a cross section of a version of the double lumen catheter that is modified such that both lumens are contained within the outer circumference of the catheter, thereby providing a smooth, even outer surface.

A number of conventional materials commonly used in the medical industry can be used to make either catheter. Examples of such materials include, but are not limited to, polyvinyl chloride, polyethylene, polypropylene, polyethylene terephthalate, polyurethane, polytetrafluoroethylene, fluoroethylenepropylene, or nylon, or combinations thereof. Examples of suitable materials are disclosed, e.g., in U.S. Pat. Nos. 6,165,166; 4,707,389, 3,561,493. The structural properties of the subject cautery device and catheter will be dictated by the intended use. For example, use of the subject cautery device with a flexible endoscope will require that the catheter is also flexible. Those skilled in the art will readily recognize appropriate materials for making such catheters to meet this requirement, as well as in the case where there is a need for a more rigid catheter.

The subject cautery device has a number of applications, in a number of different medical disciplines. With respect to gastroenterology, the subject invention may be useful to treat, for example, vascular malformations, watermelon stomach, gastric antral vascular ectasias, radiation injury, benign neoplasms, post-polypectomy bleeding, post-endoscopic ampullary sphincterotomy bleeding, ulcers, Dieulafoy's lesions, malignant neoplasms, Barrett's esophagus with or without dysplasia, varices, bleeding Mallory-Weiss tears, as well as to ablate malignant or hemorrhagic neoplasms. Additionally, it may be used to abate bleeding from portal hypertensive gastropathy or colitis, or for fistula occlusion. With respect to urology, the subject invention may be useful to treat, for example, chronic bleeding associated with retropubic prostatectomy, transurethral resection of the prostate, and other complications associated with urogenital surgical procedures. With respect to gynecology, the subject invention may be useful in treating lesions in the endocervical canal.

Alternatively, the procedure may be used as a safe effective method of contraception. Over the past 30 years, interest in population control and personal choice has led to a dramatic increase in the use of contraceptive methods, including voluntary sterilization, insertion of intra-uterine devices (IUD), administration of pills, and insertion of implants to avoid unplanned or unwanted childbearing. Female sterilization has become the most prevalent method of fertility regulation accounting for one-third of all contraceptive use worldwide. (Cooper J. Clinical Obstetrics and Gynecology 1992 35(2) 282-298). However, traditional tubal sterilization methods have been implicated in maternal death because the risky procedure often requires major surgery, anesthesia or both. (Shuber, J Am J Obstet Gynecol 1989 April: 160(4): 887-889) The present invention addresses the need for a safer, simpler non-surgical, non-hormonal sterilization procedure. Through direct application of a caustic sclerosing substance, complete tubal occlusion in an outpatient setting may be achieved. The efficacy of such a procedure has been studied in humans and animals. Shuber. J (Am J Obstet Gynecol 1989 April: 160(4):887-889) administered Methyl 2-cyanoacrylate to the uterocomual tubal junction in 35 healthy, parous women. Hysterosalpingography 4 months after the procedure showed bilateral tubal occlusion in 88.2% of the study participants. No complications were reported and there were no pregnancies reported in those participants who demonstrated tubal occlusion. In another study, the uterotubal junction (UTJ) in rabbits was destroyed using bipolar electrical current, and a plug containing either quinacrine or platelet extract was inserted. Histologic assessment by serial sections indicated occlusion of the UTJ in 96% of the rabbits treated.(Vancaillie, TG et al. Fertil Steril 1989 February, 51(2):335-8) The present method differs from these two examples in the materials used and the mode of application, potentially making it a superior method of sterilization.

Examples of caustic agents appropriate for use with the teachings herein include, but are not limited to, silver nitrate, zinc chloride, copper sulfate, phenol, acids, alkali, iodine, absolute alcohol, potassium permanganate, formalin\or combinations thereof. Furthermore, depending on the intended use, the viscosity and strength or concentration of the selected caustic agent is routinely adjusted. Where deeper penetration of the caustic agent is preferred, a more concentrated solution of the caustic agent should be used. Other characteristics such as speed and severity of cautery are adjusted as well, depending on the desired use and may be achieved by altering viscosity.

The activity of the caustic agent is readily controlled by using silver compounds such as silver nitrate and silver thiocyanate or other compounds which can release silver ions. The silver ions react with the sulfides, proteins, and chlorides in cells. Since the sulfides and chlorides are vital to cell metabolism, the reaction results in necrosis of the cells. Another potentially useful agent is iodine, which is radiopaque like silver. Compositions containing iodine react with the target tissue as the result of the release of elemental free Iodine and the reaction can be stopped by forming a stable compound, for example, sodium iodide by instilling sodium chloride. In an especially preferred embodiment, silver nitrate and DEXTRAN 70® are utilized together because they are easy to work with, are controllable, and are recognized by the medical profession and government regulatory agencies as acceptable agents for human use. DEXTRAN 40® and 70® can be used intravenously and intramuscularly and in several organ systems such as the genital tract. Silver nitrate is used on the skin, upper respiratory tract, lower genital tract, and other locations. The silver ion has a loose but stable binding with the dextran carrier but is pulled off by the consumption of the ion at the tissue sites by binding to anions and protein. The carrier may be made of dextrans or glucose or other sugars used in intravenous solutions but preferably in concentrations sufficient to form gels or pastes. The compositions prepared in accordance with this invention have a viscosity that is suitable for their intended purpose at temperatures between about 20° C. and about 37° C., however, the viscosity may be adjusted as specific applications dictate. Alginates, aloe, carboxymethylcellulose, silicones and oxidized cellulose may also be used to form pastes and gels but the dextrans and sugars are the preferred choices because of their acceptance by the medical profession and regulatory agencies. Alternatively, the practitioner may use formalin as an inexpensive, effective treatment to control bleeding. Several studies have demonstrated the efficacy of topical formalin application in the treatment of hemorrhagic radiation induced proctitis to control bleeding (Seow-Choen, F. et al. Dis Colon Rectum, 1993 February 36(2)135-36; Saclarides,T. Dis Colon Rectum 1996 February 39(2):196-199)

The speed and severity of the chemical necrosis may be regulated by the percentage of the silver nitrate in the paste. By increasing the percentage of the silver nitrate in the paste the possibility for a deeper burn is increased. It is possible, by procedures well known to those skilled in the art, to determine the appropriate concentration of silver nitrate to achieve the desired depth of cauterization for specific applications. The practitioner may readily formulate a paste that is essentially self-regulating. For example, a weak silver nitrate paste may be formulated that will expend itself after necrosing to a depth of only half the maximum safely allowable depth, thereby reducing the danger of necrosing too deeply. Preferably, the composition comprises 1-50%, by weight, of caustic agent. More preferred, the caustic agent comprises 10-40%, by weight of the composition. Alternatively, the practitioner may easily terminate the treatment by introducing a normal saline solution, e.g., NaCl, which will deactivate the silver nitrate by forming silver chloride. An advantage of the silver nitrate is that the deactivating agent for the silver ion is the chloride ion found in several solutions used regularly in medicine, e.g., intravenously and intramuscularly, such as normal saline or Ringer's solution. The silver nitrate deactivation is the essentially stoichiometric formation of an insoluble non-caustic precipitate. The viscosity of the caustic composition may be adjusted so that it does not flow uncontrollably from the site of need. The caustic composition should flow easily, i.e, without excessive pressure, through a catheter having an inside diameter of about 1-2 mm. Preferably, the caustic composition should be thick enough that it does not run, i.e., it stays in the vicinity of the point of application. In a preferred embodiment, a caustic composition having a consistency ranging from toothpaste to pancake syrup is utilized as specific applications dictate. The ability to use a desired consistency will be limited only by the internal dimensions of the catheter employed. Thixotropic caustic compositions utilizing, e.g., mineral clays or the like may be especially useful in some applications. While modifying the viscosity of the cauterizing compound can alter the flow properties and therefore aid in the control of delivery, the subject cautery device allows for controlled delivery of a cauterizing agent having a broad range of viscosities as result of its regulating tip.

The teachings of all patents and publications cited throughout this specification are incorporated by reference in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A cautery device comprising:
a). a catheter comprising a first end and a second end, wherein said first end comprises a regulating tip to govern delivery of a caustic agent from said catheter to a site of need; and
b). a container for supplying said caustic agent to said catheter attached to said second end.

2. The cautery device of claim 1, wherein said regulating tip comprises a liquid permeable material positioned therein to regulate flow of said caustic agent.

3. The cautery device of claim 2, wherein said liquid permeable material comprises a sponge, fritted glass, semi-permeable membrane, or combinations thereof.

4. The cautery device of claim 1, wherein said regulating tip comprises a closed end having one or more holes defined thereon.

5. The cautery device of claim 1, wherein said catheter is flexible.

6. The cautery device of claim 1, wherein said container comprises a syringe or pump.

7. The cautery device of claim 1 wherein said caustic agent comprises silver nitrate, zinc chloride, copper sulfate, phenol, acids, alkali, iodine, potassium permanganate, absolute alcohol, formalin, or combinations thereof.

8. The cautery device of claim 1 wherein said catheter is comprised of polyvinyl chloride, polypropylene, polyethylene, polyethylene terephthalate, polyurethane, polytetrafluoroethylene, fluoroethylenepropylene or nylon, or combinations thereof.

9. The cautery device of claim 1 wherein said regulating tip comprises an impeder which governs the flow of delivery of said caustic agent to the site of need as to limit undesired spillage of said caustic agent than without said impeder, thereby avoiding damage to tissues surrounding the site of need.

10. The cautery device of claim 1, wherein said regulating tip is comprised of a flexible material having a slit formed therein.
